(19) United States
(12) Patent Application Publication
(10) Pub. No.: US 2017/0354551 A1
(43) Pub. Date: Dec. 14, 2017

(54) HYGIENE ARTICLES WITH HIGH FRICTION SURFACES AND METHODS OF MAKING AND USING THEM

(71) Applicants: Peter Gawley, Roanoke, VA (US); Jens Soder, Gottingen (DE)

(72) Inventors: Peter Gawley, Roanoke, VA (US); Jens Soder, Gottingen (DE)

(21) Appl. No.: 15/439,632

(22) Filed: Feb. 22, 2017

Related U.S. Application Data
(63) Continuation of application No. 14/078,145, filed on Nov. 12, 2013, now abandoned.
(60) Provisional application No. 61/725,969, filed on Nov. 13, 2012.

(51) Int. Cl. A61F 13/514; A61F 17/00; A61F 13/36

(52) U.S. Cl. CPC A61F 13/51484; A61F 13/36; A61F 17/00

(57) ABSTRACT

Certain embodiments described herein are directed to hygiene articles that comprise a coefficient of friction of greater than 0.95 as tested by ASTM D 1894. In some embodiments, the article comprises an absorbent core material disposed on a backing layer that is effective to provide a coefficient of friction of greater than 0.95 as tested by ASTM D 1894.

### PRIORITY APPLICATION

This application claims priority to U.S. Application No. 61/725,969 filed on Nov. 13, 2012, the entire disclosure of which is hereby incorporated herein by reference for all purposes.

### TECHNOLOGICAL FIELD

Certain features, aspects and embodiments are directed to hygiene articles including or one or more high friction surfaces. In certain embodiments, an absorbent pad comprising at least one high friction surface is provided.

### BACKGROUND

Hygiene articles are available in many different configurations. Hygiene articles may be configured for various uses including commercial and personal uses.

### SUMMARY

In one aspect, an article comprising an absorbent core material disposed on a backing layer, in which the backing layer comprises at least one area comprising a coefficient of friction that is greater than 0.95 as tested by ASTM D 1894, e.g., ASTM D1894 dated 2008, is provided.

In certain embodiments, the article comprises an effective tear strength to be removed from under an object, weighing about 325 lbs or more, on the article without substantial tearing of the article. In other embodiments, the article can be repositioned under an object weighing about 325 lbs or more without substantial tearing of the article. In some examples, the backing layer comprises a non-woven material effective to provide the effective tear strength to the article. In other embodiments, the absorbent core material comprises at least one superabsorber. In some examples, the article also comprises a top layer disposed on the absorbent core material. In some embodiments, the top layer comprises a polypropylene material. In certain examples, at least one section of the absorbent core material is exposed to the environment. In other examples, the article further comprises a tissue paper layer covering adjacent sections of the absorbent core material. In some embodiments, the backing layer comprises two substantially parallel strips to provide the coefficient of friction that is greater than 0.95 as tested by ASTM D 1894. In other embodiments, the entire backing layer comprises a unitary sheet effective to provide the coefficient of friction that is greater than 0.95 as tested by ASTM D 1894.

In an additional aspect, an article comprising an absorbent core material disposed on a backing layer, in which the article is sized and arranged to be placed under a human to absorb bodily fluids from the human and in which the article comprises at least one material disposed on the backing layer that is effective to provide a coefficient of friction that is greater than 0.95 as tested by ASTM D 1894 is described.

In certain embodiments, the at least one material disposed on the backing layer is configured as a plurality of strips. In some embodiments, the at least one material disposed on the backing layer is configured as a sheet. In other embodiments, the absorbent core material comprises at least one superabsorber. In some examples, the article further comprises a top layer disposed on the absorbent core material. In additional examples, the top layer comprises a polypropylene material. In other examples, at least one section of the absorbent core material is exposed to the environment. In further embodiments, the article further comprises a tissue paper layer covering adjacent sections of the absorbent core material. In some embodiments, the at least one material disposed on the backing layer comprises a plurality of offset strips. In other embodiments, the at least one material disposed on the backing layer comprises at least two areas with different coefficients of frictions greater than 0.95 as tested by ASTM D 1894.

In another aspect, an article comprising a backing layer comprising an effective amount of a material to provide a coefficient of friction that is greater than 0.95 as tested by ASTM D 1894, an effective amount of an absorbent core material disposed on the backing layer to absorb at least 250 mL of fluid without substantial leakage of the fluid, and a top layer disposed on the absorbent core material is disclosed.

In certain embodiments, the article comprises an effective tear strength to be removed from under an object, weighing about 325 lbs or more, on the article without substantial tearing of the article. In other embodiments, the backing layer comprises a non-woven material effective to provide the effective tear strength to the article. In some examples, the absorbent core material comprises at least one superabsorber. In additional examples, the article further comprises a top layer disposed on the absorbent core material. In other examples, the top layer comprises a polypropylene material. In some embodiments, at least one section of the absorbent core material is exposed to the environment. In other examples, the article further comprises a tissue paper layer covering adjacent sections of the absorbent core material. In some embodiments, the backing layer comprises two substantially parallel strips to provide the coefficient of friction that is greater than 0.95 as tested by ASTM D 1894. In additional embodiments, the entire backing layer comprises a unitary sheet effective to provide the coefficient of friction that is greater than 0.95 as tested by ASTM D 1894.

In an additional aspect, a backing layer comprising an effective amount of a material to provide a coefficient of friction that is greater than 0.95 as tested by ASTM D 1894, an effective amount of an absorbent core material on the backing layer and constructed and arranged to be placed under the body of a human subject weighing at least 325 lbs and absorb at least 250 mL of body fluid from the human subject without substantial leakage of the body fluid, and a top layer disposed on the absorbent core material, in which the article comprises an effective tear strength to be removed from under the human subject weighing at least 325 lbs or more by pulling the article from being under the human subject and without substantial tearing of the article during removal. In some embodiments, the article may be effective to permit repositioning of the article under the human subject weighing at least 325 lbs without substantial tearing of the article.

In certain examples, the backing layer comprises a non-woven material effective to provide the effective tear strength to the article. In other embodiments, the absorbent core material comprises at least one superabsorber. In further examples, the article comprises a pattern in one or more of the core material and the top layer. In some examples, the top layer comprises a polypropylene material. In other examples, at least one section of the absorbent core material is exposed to the environment. In some examples, the article comprises a tissue paper layer covering adjacent sections of the absorbent core material. In certain embodiments, the backing layer comprises a plurality of individual strips to provide coefficient of friction that is greater than 0.95 as tested by ASTM D 1894. In some embodiments, the backing layer comprises two substantially parallel strips to provide the coefficient of friction that is greater than 0.95 as tested by ASTM D 1894. In additional embodiments, the entire backing layer comprises a unitary sheet effective to provide the coefficient of friction that is greater than 0.95 as tested by ASTM D 1894.

In another aspect, a packaging material comprising a backing layer and an absorbent core material disposed on the backing layer, in which the article comprises a coefficient of friction that is greater than 0.95 as tested by ASTM D 1894 is disclosed.

In certain embodiments, the absorbent material comprises one or more superabsorbers. In other embodiments, the packaging material further comprises a top layer disposed on the absorbent material. In some embodiments, the backing layer comprises a plurality of individual strips to provide coefficient of friction that is greater than 0.95 as tested by ASTM D 1894. In other examples, the backing layer comprises at least one strip of material that provides the coefficient of friction that is greater than 0.95 as tested by ASTM D 1894. In some examples, the backing layer comprises a plurality of strips sized different from each other and each effective to provide the coefficient of friction that is greater than 0.95 as tested by ASTM D 1894. In other embodiments, the backing layer comprises two substantially parallel strips to provide the coefficient of friction that is greater than 0.95 as tested by ASTM D 1894. In some examples, the entire backing layer comprises a unitary sheet effective to provide the coefficient of friction that is greater than 0.95 as tested by ASTM D 1894. In additional examples, the packaging material further comprises a material disposed on the absorbent core material that is effective to provide a coefficient of friction that is greater than 0.95 as tested by ASTM D 1894. In some instances, the disposed material is configured as at least one strip.

In an additional aspect, a tuckable sheet sized and arranged for placement on a hospital bed, the tuckable sheet comprising a backing layer effective to provide a coefficient of friction that is greater than 0.95 as tested by ASTM D 1894, and an absorbent core material disposed on the backing layer is provided.

In certain embodiments, the absorbent material comprises one or more superabsorbers. In other embodiments, the tuckable sheet further comprises a top layer disposed on the absorbent material. In some examples, the tuckable sheet further comprises at least one attachment means configured to retain the tuckable sheet to the hospital bed. In certain examples, the backing layer comprises at least one strip of material that provides the coefficient of friction that is greater than 0.95 as tested by ASTM D 1894. In some embodiments, the backing layer comprises a plurality of individual strips to provide the coefficient of friction that is greater than 0.95 as tested by ASTM D 1894. In other embodiments, the backing layer comprises two substantially parallel strips to provide the coefficient of friction that is greater than 0.95 as tested by ASTM D 1894. In certain examples, the entire backing layer comprises a unitary sheet effective to provide the coefficient of friction that is greater than 0.95 as tested by ASTM D 1894. In other examples, the tuckable sheet further comprises a material disposed on the absorbent core material that is effective to provide a coefficient of friction that is greater than 0.95 as tested by ASTM D 1894. In some embodiments, the disposed material is configured as at least one strip.

In another aspect, a gurney pad sized and arranged to substantially cover the top surface of a gurney, the gurney pad comprising a backing layer effective to provide a coefficient of friction that is greater than 0.95 as tested by ASTM D 1894, and an absorbent core material disposed on the backing layer is described.

In certain embodiments, the gurney pad is effective to absorb moisture from a patient that weighs about 325 lbs or more placed on the gurney and comprises an effective tear strength to be removed from under the patient that weighs about 325 lbs or more on the gurney without substantial tearing of the gurney pad. In some examples, the absorbent material comprises one or more superabsorbers. In other examples, the gurney pad comprises a top layer disposed on the absorbent material. In some embodiments, the backing layer comprises at least one strip of material that provides the coefficient of friction that is greater than 0.95 as tested by ASTM D 1894. In other embodiments, the backing layer comprises a plurality of individual strips to provide the coefficient of friction that is greater than 0.95 as tested by ASTM D 1894. In further embodiments, the backing layer comprises two substantially parallel strips to provide the coefficient of friction that is greater than 0.95 as tested by ASTM D 1894. In certain embodiments, the entire backing layer comprises a unitary sheet effective to provide the coefficient of friction that is greater than 0.95 as tested by ASTM D 1894. In other embodiments, the gurney pad comprises a material disposed on the absorbent core material that is effective to provide a coefficient of friction that is greater than 0.95 as tested by ASTM D 1894. In certain embodiments, the disposed material is configured as at least one strip.

In another aspect, an operating table pad sized and arranged to substantially cover the top surface of an operating table, the pad comprising a backing layer effective to provide a coefficient of friction that is greater than 0.95 as tested by ASTM D 1894, and an absorbent core material disposed on the backing layer is disclosed.

In certain embodiments, the operating table pad is effective to absorb moisture from a patient that weighs about 325 lbs or more placed on the operating table and comprises an effective tear strength to be removed from under the patient that weighs about 325 lbs or more on the operating table without substantial tearing of the pad. In some examples, the absorbent material comprises one or more superabsorbers. In other examples, the operating room table pad comprises a top layer disposed on the absorbent material. In some embodiments, the backing layer comprises at least one strip of material that provides the coefficient of friction that is greater than 0.95 as tested by ASTM D 1894. In other embodiments, the backing layer comprises a plurality of individual strips to provide the coefficient of friction that is greater than 0.95 as tested by ASTM D 1894. In further embodiments, the backing layer comprises two substantially parallel strips to provide the coefficient of friction that is greater than 0.95 as tested by ASTM D 1894. In certain embodiments, the entire backing layer comprises a unitary sheet effective to provide the coefficient of friction that is greater than 0.95 as tested by ASTM D 1894. In other embodiments, the operating room table pad comprises a material disposed on the absorbent core material that is effective to provide a coefficient of friction that is greater than 0.95 as tested by ASTM D 1894. In certain embodiments, the disposed material is configured as at least one strip.

In an additional aspect, a kit comprising a backing layer effective to provide a coefficient of friction that is greater than 0.95 as tested by ASTM D 1894, an absorbent core material, and instructions for coupling the absorbent core material to the backing layer to provide an article is described.

In certain examples, the backing layer comprises at least one strip of material that is effective to provide the coefficient of friction that is greater than 0.95 as tested by ASTM D 1894. In some examples, the kit further comprises at least one adhesive effective to couple the core material and the backing layer. In other examples, the kit further comprises at least one top layer material configured to couple to the core material. In some embodiments, the absorbent core material comprises at least one superabsorbent material. In certain examples, the kit further comprises an additional backing layer different from the backing layer. In other embodiments, the kit comprises an additional absorbent core material different from the absorbent core material. In some examples, the backing layer comprises two substantially parallel strips to provide the coefficient of friction that is greater than 0.95 as tested by ASTM D 1894. In certain embodiments, the entire backing layer comprises a unitary sheet effective to provide the coefficient of friction that is greater than 0.95 as tested by ASTM D 1894. In some examples, the kit further comprises a material configured to couple to the absorbent core material that is effective to provide a coefficient of friction that is greater than 0.95 as tested by ASTM D 1894.

In another aspect, a kit comprising a backing layer, an absorbent core material, a material configured to couple to the backing layer or the absorbent core material or both to provide a coefficient of friction that is greater than 0.95 as tested by ASTM D 1894, and instructions for coupling the absorbent core material to the backing layer to provide an article is disclosed.

In certain embodiments, the backing layer comprises at least one strip of material that is effective to provide the coefficient of friction that is greater than 0.95 as tested by ASTM D 1894. In some examples, the kit further comprises at least one adhesive effective to couple the core material and the backing layer. In other embodiments, the kit further comprises at least one top layer material configured to couple to the core material. In some examples, the absorbent core material comprises at least one superabsorbent material. In additional examples, the kit further comprises an additional backing layer different from the backing layer. In some examples, the kit further comprises an additional absorbent core material different from the absorbent core material. In some embodiments, the material comprises at least one strip to provide the coefficient of friction that is greater than 0.95 as tested by ASTM D 1894. In additional embodiments, the material is configured as a unitary sheet effective to provide the coefficient of friction that is greater than 0.95 as tested by ASTM D 1894. In further examples, the material is configured as a plurality of strips each configured to provide the coefficient of friction that is greater than 0.95 as tested by ASTM D 1894.

In an additional aspect, a method of facilitating production of an article, the method comprising providing a backing layer effective to provide a coefficient of friction that is greater than 0.95 as tested by ASTM D 1894, and providing instructions for using the backing layer with a core material to provide an article is provided.

In certain examples, the method comprises providing the core material. In further examples, the method comprises providing a top layer material configured to couple to the core material. In some examples, the method comprises providing instructions for forming a pattern in the article. In additional examples, the method comprises providing an antimicrobial material for applying to the article. In other embodiments, the method comprises configuring the article to comprise an effective tear strength to be removed from under a patient weighing about 325 lbs or more without substantial tearing of the article. In other examples, the method comprises providing a plurality of different core materials. In further examples, the method comprises providing a plurality of different backing layers each effective to provide the coefficient of friction that is greater than 0.95 as tested by ASTM D 1894. In additional examples, the provided backing layer comprises a strip of material that provides the coefficient of friction that is greater than 0.95 as tested by ASTM D 1894. In other examples, the provided backing layer comprises a unitary sheet that provides the coefficient of friction that is greater than 0.95 as tested by ASTM D 1894.

In some aspects, the articles, gurney pads, tuckable sheets, and kits described herein can be used with an antimicrobial material disposed on the article, e.g., disposed on the core material.

Additional features, aspects and examples are described in more detail below.

### BRIEF DESCRIPTION OF THE FIGURES

Certain illustrative embodiments are described in more detail below with reference to the accompanying figures in which:

FIG. 1 is a cross-section illustration of one embodiment of an article comprising a backing layer and a core material layer, in accordance with certain examples;

FIG. 2 is an illustration of an article including a strip of material with a high coefficient of friction, in accordance with certain examples;

FIG. 3 is another illustration of an article including a strip of material with a high coefficient of friction, in accordance with certain examples;

FIG. 4 is an illustration of an article including two strips of material with a high coefficient of friction, in accordance with certain examples;

FIG. 5 is another illustration of an article including two strips of material with a high coefficient of friction, in accordance with certain examples;

FIG. 6 is an illustration of an article including a plurality of areas of material with a high coefficient of friction, in accordance with certain examples;

FIG. 7 is a top view of an embodiment of an article comprising a plurality of individual articles that can be separate from each other, in accordance with certain examples; and

FIG. 8 is a top view of another embodiment of an article comprising a backing layer, a core material layer and a top layer, in accordance with certain examples.

It will be recognized by the person of ordinary skill in the art, given the benefit of this disclosure, that the relative positions and sizes of the components in the figures are not limiting and that no particular size, dimension, thickness or arrangement is implied as being required based on the representations of the components shown in the figures. Where a particular geometric representation is shown, the representation is provided for illustrative purposes only and is not intended to imply that the articles and other devices and components necessarily take that particular representation for proper function or usage.

### DETAILED DESCRIPTION

In the illustrative embodiments described below, certain components are included in the articles. Depending on the desired configuration of the article, it may be desirable to include additional components, omit one or more components or rearrange or substitute other components into the articles. Unless otherwise specified the article can be a single article or can be coupled to additional articles, e.g., in sheet form, which can be separated prior to packaging or may be separated by an end user prior to, during or after use. In some embodiments, the articles can be coupled to each other through one or more perforations to provide for easy separation. The articles can be stacked, folded, festooned or otherwise coupled to each other or packaged in a desired manner for a particular use.

In certain embodiments, the articles described herein can be used in many different applications where it is desirable to absorb, retain or both a fluid or substance. For example, the article can be configured or designed to retain a body fluid or material such as urine, breast milk, saliva, fecal matter, sweat, mucus, vaginal secretions or other bodily fluids or materials commonly excreted or expelled by a human. The non-skid nature of certain embodiments provided herein expand use of the absorbent articles as floor pads in operating rooms, emergency rooms and other settings where body fluids may end up on the floor or surfaces and create a slip hazard.

In addition to the absorptive properties provided by embodiments of the articles described herein, the increased friction of the articles permits their use on slick surfaces or on surfaces where it may be desirable to retain the article in place without the use of a tape, adhesive or other fastener. In some embodiments, an article may be configured as an absorbent pad, e.g., a sanitary pad to absorb urine, and placed on a desired surface such as a hospital bed, operating table, operating room floor, emergency room floor or other places as described herein. A patient may be placed on the sanitary pad. The sanitary pad can subsequently be used to absorb any urine or other fluids from the patient. It is a substantial attribute of the articles described herein that they can be used in a non-skid fashion to absorb fluids without the risk of the pad sliding unwantedly.

In certain embodiments, reference is made herein to ASTM D 1894 which refers to a standard ASTM test entitled “Standard Test Method for Static and Kinetic Coefficients of Friction of Plastic Film and Sheeting.” For example, ASTM D 1894 dated 2008 can be used to determine the coefficient of friction (COF) of the articles described herein.

In certain examples, the articles described herein can be configured to provide increased tear strength compared to existing articles. In some embodiments, the article can be configured for use with objects weighing more than 325 lbs without tearing of the article. For testing purposes, an object weighing more than about 325 lbs can be placed on an article. The article may then be pulled away from being under the object without tearing of the article. In certain embodiments, the article can be configured to accept an object weighing 350 lbs or more, 375 lbs or more, 400 lbs or more, 425 lbs or more, 450 lbs or more, 475 lbs or more, or 500 lbs or more and can be removed without substantial tearing after the object is placed on the article. In other embodiments, the article can have a tear strength (as measured ASTM D882 dated 2002) of at least about 50 N/25 mm (MD) and 35 N/25 mm (CD), more particularly about 100 N/25 mm (MD) and 50 N/25 mm (CD), more particularly, at least 133 N/25 mm (MD) and 73 N/25 mm (CD), e.g., the article can be configured to provide a tear strength of at least MD 140 N/25 mm (MD) and at least 75 N/25 mm (CD).

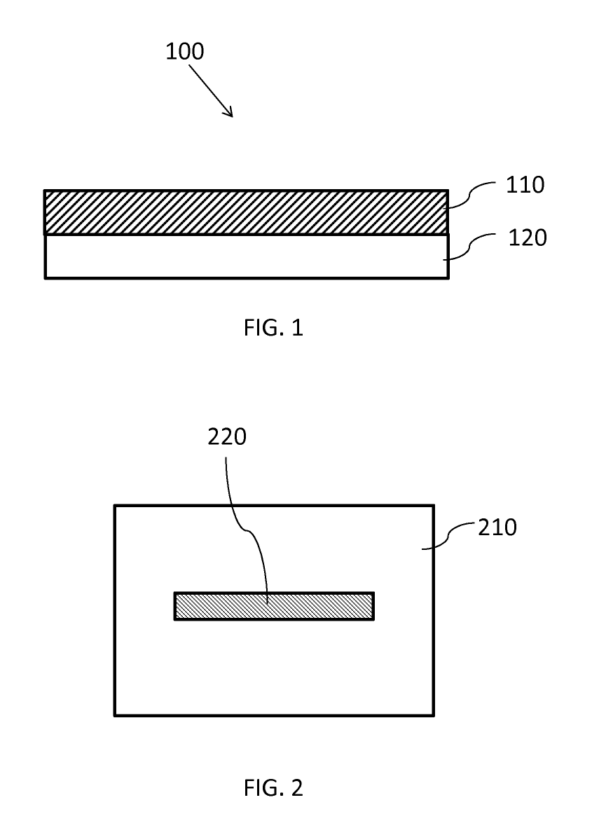

In some examples and referring to FIG. 1, an article 100 including a core layer 110 and a backing layer 120 is shown. The term layer in reference to FIG. 1 is used herein for convenience purposes only and the various materials used in the article 100 can be configured as a film, sheet, patches or take other forms. In addition, while the layers are shown for illustration purposes in FIG. 1 as having the same thickness, it will be recognized by the person of ordinary skill in the art, given the benefit of this disclosure, that the layers 110 and 120 typically will not have the same thickness. Illustrative thicknesses and dimensions for each layer are provided herein as representative sizes of the many different dimensions that can be present in the articles described herein. In use of the article 100, the core layer 110 typically contacts an object (not shown) and is effective to absorb fluid from the object. The backing layer 120 typically provides a supportive structure to the core layer 110 and assists in increasing the overall tear strength for the article 100. In some embodiments, the backing layer 120 may be configured as a material that is effective to provide a coefficient of friction of greater than 0.95 as tested by ASTM D 1894. In other embodiments, the backing layer 120 may comprise an area or portion that provides the overall coefficient of friction of 0.95 of greater, more particularly about 1.0 or greater, 1.05 or greater, 1.1, 1.2, 1.3, 1.4, 1.5 or 2.0 or greater as tested by ASTM D 1894. In some instances described herein, the backing layer 120 may comprise strips of material that provide the coefficient of friction of greater than 0.95 as tested by ASTM D 1894. While not wishing to be bound by any particular scientific theory, the ASTM D 1894 test uses a material of a specified size. The material is taped to a plate, and a sled is added on top of the material. A string is connected to the sled, and a force is applied until the sled begins to move. The force required to move the sled correlates to the coefficient of friction. When more force is required to move the sled it is indicative of a higher coefficient of friction. The force required to initially move the sled is referred to as the static coefficient of friction. The sled may be moved over a specified distance to determine the kinetic coefficient of friction. Unless otherwise specified herein and in the appended claims, the values of the coefficient of friction refer to the static coefficient of friction values.

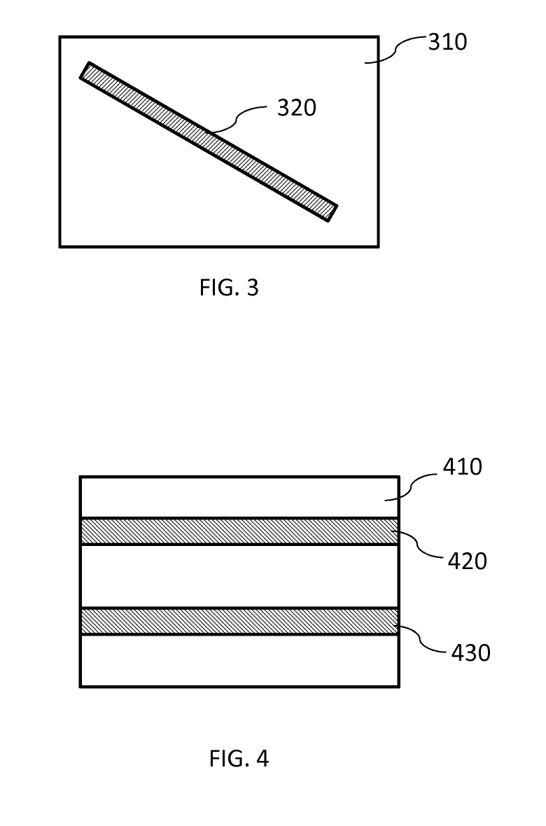

In certain embodiments, the backing sheet may include one or more areas or portions of material that provide the desired coefficient of friction. Referring to FIG. 2, a bottom view of an article shows a backing sheet 210 comprising a single strip 220 of a material that is effective to provide the coefficient of friction of about 0.95 or greater to the article. The strip 220 of material can be positioned along a direction generally parallel to the longitudinal direction of the backing layer 210 or may be positioned in other angles. For example and referring to FIG. 3, a strip 320 is shown as being position diagonally on a backing layer 310. If desired, the strip may be discontinuous such that one or more sharp angles exist in the strip, e.g., a ninety degree angle or other angle. The strip 220 or 320 is generally effective to engage an underlying surface and prevent movement of the article comprising the backing layer 210 or 310. In some embodiments, the strip 220 or 320 may be covered with a release material, layer or liner to permit placement and movement of the backing layer 210 or 310 until it is positioned at a desired site. The release layer or liner can then be removed and the strip 220 or 320 can engage the surface and generally deter movement of the backing layer on the surface. While the strip 220 and 320 is shown in FIGS. 2 and 3 as being a separate material from the backing layer 210 or 310, it may be integral to the backing layer 210 or 310 or may be part of a film or sheet that is added to the backing layer 210 or 310 and generally overlies the backing layer 210 or 310.

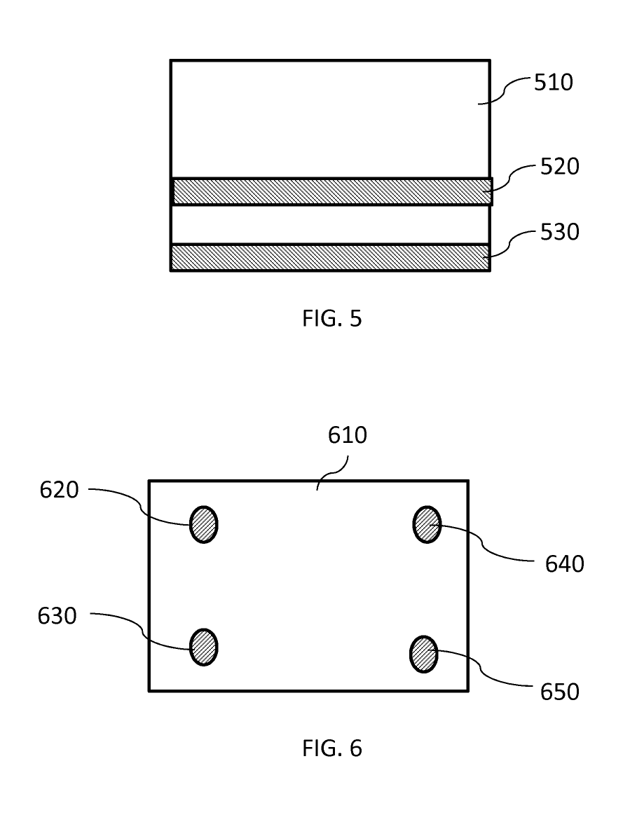

In certain examples, more than a single strip can be positioned on the backing layer or the backing layer can be configured with two or more integral areas that provide the high friction areas. For example and referring to FIG. 4, a backing layer 410 is shown as comprising a first strip 420 and a second strip 430. The strips 420 and 430 are generally parallel to each other and run from one side of the backing layer 410 to the other. While the strips 420 and 430 are generally shown to be positioned at roughly the same distance from the sides of the backing layer 410, such positioning is not required. In some embodiments, the strips can be offset. For example and referring to FIG. 5, a backing layer 510 comprises a first strip 520 and a second strip 530 that is offset from the first strip 520, e.g., is spaced a different distance from one or more sides of the backing layer 510.

In certain examples, the exact length and/or width of the strips shown in FIGS. 2-5 can vary. In some embodiments, the length of the strips may be about the same as the length of the backing layer or up to about 5% larger than the length of the backing layer. In other embodiments, the length of the strips may be less than that of the backing layer. In further embodiments, the length of the strips may be about 75% of the length of the backing layer, about 50% of the length of the backing layer or about 25% of the length of the backing layer. In embodiments where the length of the strips is small, e.g., less than 25% of the length of the backing layer, it may be desirable to include an increased numbers of strips, e.g., 3 or more. Similarly, the width of the strips can be up to the width of the backing layer, or if desired, about 5% wider than the width of the backing layer to increase the overall contact of the high friction material with the surface. In other examples, the width of the strips may be about 75% of the width of the backing layer, about 50% of the width of the backing layer or about 25% of the width of the backing layer. The length and width of the strips need not be the same along the entire length or width of the strips. For example, the strips may include some areas that are wider than other areas.

In certain embodiments, the backing layer need not include a high friction material as a strip. Instead, small areas or portions that have a different shape than a strip shape can be used. For example, and referring to FIG. 6, a backing layer 610 is shown as comprising areas 620, 630, 640 and 650, which have a generally circular shape. The areas 620, 630, 640 and 650 can be configured to individually provide a high friction surface or in concert may provide the high friction surface, e.g., a surface with a coefficient of friction of about 0.95 or greater as measured by ASTM D 1894. While the areas 620, 630, 640 and 650 are shown as being generally circular, such geometry is not requires and other geometries such as, for example, triangular, rectangular, pentagonal, hexagonal, octagonal or other shapes can also be used. In addition, while four different areas are shown in FIG. 6, fewer than four, e.g., 1, 2 or 3, areas can be present, or more than four, e.g., 5, 6, 7, 8 or more areas can be present if desired.

In certain embodiments, the strips or areas of the backing layer that provide the high coefficient of friction may be produced using many different materials. In some embodiments, the material can be selected from one or more thermoplastic materials comprising an additive that can provide the desired coefficient of friction, e.g., an additive that can provide a COF of 0.95 or greater as tested by ASTM D 1894. In other embodiments, the strips or areas may take the form of a sheet of material that can include an additive that can provide the desired coefficient of friction. In some examples, the sheet can be produced from one or more thermoplastic materials including, but not limited to, a polyethylene, a polypropylene, a polyester-polypropylene, an acrylonitrile butadiene styrene, a cellulose acetate, an ethylene-vinyl acetate, an ethylene vinyl alcohol, a fluoroplastic such as polytetrafluoroethylene, a polyoxymethylene, a polyacrylate, a polyacrylonitrile, a polyamide, a polyamide-imide, a polyetheretherketone, a polyaryletherketone, a polybutadiene, a polybutylene, a polycaprolactone, a polychlorotrifluoroethylene, a polyetherketoneketone, a polycarbonate, a polyhydroxyalkanoate, a polyketone, a polyester, a polyetherimide, a polysulfone, a polyimide, a polylactic acid, a polymethylpentene, a polyphenylene oxide, a polyphenylene sulfide, a polyphthalamide, a polystyrene, a polyurethane, a polyvinyl acetate, a polyvinyl chloride, a polyvinylidene chloride, a styrene-acrylonitrile, combinations thereof and other suitable thermoplastics. In some embodiments, the additive may be many different materials including powders, whiskers, fillers, fibers, abrasives, or other materials that can provide a coefficient of friction of about 0.95 or greater. In some embodiments, the additive can be mixed with the thermoplastic prior to forming or blowing of the sheet or strips, whereas in other examples, the additive can be added to the sheet or strip after it has been formed. For example, the sheet or strip can be heated and the additive may be added to one surface to provide the desired COF.

In some embodiments, the backing layer itself can include a suitable basis weight, thickness, tear strength, elongation or combinations thereof to provide an article having desired properties. In some embodiments, the backing layer may have a total weight of at least 50 grams per square meter (gsm), more particularly at least 90 gsm, at least 100 gsm or at least 150 gsm. In some instances, the hydrohead of the backing layer, as measured by EN 20811 (rate of rise of 60 cm/minute) can be at least 80 cm of water, more particularly at least 100 cm of water, for example about 125 cm of water. In other embodiments, the water vapor transmission rate (WVTR) as measured using test method Edana WSP 70.6 may be at least 2500 g/m²/24 hours, more particularly about 3000 g/m²/24/hours, for example at least about 3500 g/m²/24 hours. In certain embodiments, the backing layer can have a desirable lamination strength. In some instances, the lamination strength can be greater than or equal to about 0.75 N/25 mm as tested by Edana WSP 401.0 test method, more particularly about 0.9 N/25 mm, e.g., about 1 N/25 mm or more. In other configurations, the tear strength of the backing layer in the machine direction can be at least about 100 N/25 mm as measured using test method ASTM D822, more particularly at least 125 N/25 mm, for example, at least 150 N/25 mm or at least 200 N/25 mm in the machine direction. In some configurations, the tear strength of the backing layer in the cross direction can be at least about 60 N/25 mm as measured using test method ASTM D822, more particularly at least 90 N/25 mm, for example at least 110 N/25 mm or at least 130 N/25 mm in the machine direction. In some configurations, the tear strength at 25% elongation in the machine direction of the backing layer may be at least 50 N/25 mm as tested by ASTM D822, more particularly at least 75 N/25 mm, for example at least 100 N/25 mm. In other configurations, the tear strength at 25% elongation in the cross direction of the backing layer may be at least 30 N/25 mm as tested by ASTM D822, more particularly at least 50 N/25 mm, for example at least 60 N/25 mm. In certain embodiments, the backing layer may have an elongation at break in the machine direction of at least 50% as tested by ASTM D822, more particularly at least 90%, for example, at least 120%. In some examples, the backing layer may have an elongation at break in the cross direction of at least 60% as tested by ASTM D822, more particularly at least 100%, for example at least 150%

In certain embodiments and referring to FIG. 1 again, the core material 110 in the article 100 can comprise a material effective to absorb and/or retain fluids. In some examples, the core material 110 can comprise a polar material. In other examples, the core material 110 can comprise a material comprising a plurality of hydroxyl groups. In yet other examples, the core material can comprise cellulose or cellulose-based materials. In some embodiments, the core material can comprise a fiber mat comprising cellulose fibers sized and arranged as a loose non-woven material. In some embodiments, the core material can comprise a basis weight of about 100 grams/square meter to about 175 grams/square meter, e.g., about 110 g/square meter to about 150 grams/square meter.

In certain examples, the core layer 110 of the articles described herein can be effective to absorb and/or retain fluids or other materials. For example, the material of the core layer 110 can be selected such that fluid that contacts the article 100 becomes absorbed and trapped by the core layer 110. Once the core layer 110 becomes saturated (or after a desired period), the article 100 can be removed and replaced with a new article to absorb additional fluid. In some embodiments, the material of the core layer 110 can be selected to provide a wicking action such that fluid which contacts a certain area of the article 100 diffuses or spreads out through the core layer 110 to increase the overall fluid capacity of the article 100.

In certain instances, the physical and mechanical properties of the core layer can be selected to provide desired properties to the article. In some configurations, the basis weight of the core can be from about 80 gsm to about 160 gsm, more particularly about 100 gsm to about 150 gsm, for example, about 110 gsm to about 140 gsm. In certain embodiments, the basis weight of the core can be from one or more different materials present in the core. For example, where an absorbent material such as a superabsorber is present, the absorbent material may be present from around 5 gsm to about 50 gsm, more particularly about 10 gsm to about 40 gsm, for example about 25-35 gsm or 30 gsm. In some embodiments, the core may also include tissue, pulp or other types of paper or cellulose based material that can be present, for example, from about 20 gsm to about 100 gsm. Where tissue is present, the tissue can be present at a basis weight of about 15 gsm to about 60 gsm, more particularly about 25 gsm to about 50 gsm, e.g., about 30 gsm to about 45 gsm. Where pulp is present in the core, the pulp can be present from about 30 gsm to about 80 gsm or from about 40 gsm to about 70 gsm, for example about 45 gsm to about 65 gsm. The particular thickness of the core material may also vary depending on the desired use of the article. In some embodiments, the core material can be about 0.4 mm to about 2 mm thick, more particularly about 0.7 mm to about 1.5 mm thick, for example, about 0.8 mm thick to about 1 mm thick.

In certain instances, the core material can have a tensile strength of about 0.3 kN/m to about 0.6 k N/m, for example about 0.4 kN/m to about 0.5 kN/m. To measure the tensile strength, a method such as EDANA 20.2-89 or ISO 9073-3:1989 can be used. In some configurations, the elongation of the core (when dry) can be about 10% to about 30%, more particularly about 10% to about 20%, e.g., about 15%. In certain embodiments, the tensile strength of the core (when wet) can be from about 0.075 kN/m to about 0.15 kN/m, more particularly about 0.08 kN/m to about 0.12 kN/m, e.g., about 0.09 kN/m. In certain examples, the elongation of the core (when wet) can be about 4% to about 15%, more particularly about 4% to about 12%, for example, about 5-8% or about 7%. The thickness of the core can vary from about 0.5 mm to about 1.5 mm, more particularly about 0.65 mm to about 1 mm, for example about 0.8 mm. The thickness of the core can be measured, for example, using calipers or by determining the distance the core offsets a flat plate when the core is placed under the plate. The density of the core may vary depending on the intended use of the article and illustrative densities include, but are not limited to, about 120 kg/m³ to about 200 kg/m³, more particularly about 130 kg/m³ to about 180 kg/m³, for example, about 150 kg/m³ to about 160 kg/m³. Density may be determined by weighing a material sample and dividing by its volume. The stiffness of the core can vary from about 80 mm to about 125 mm, for example about 85 mm to about 115 mm, e.g., about 95 mm, 100 mm or 105 mm. Stiffness may be determined using the EDANA 50.5-99 test, for example.

In some embodiments, the absorption time of the core material can vary from about 4 seconds to about 16 seconds, more particularly about 6 seconds to about 12 seconds, for example, about 7, 8, 9, 10, or 11 seconds or any value in between these illustrative values. Absorption time can be measured, for example, using EDANA 153.0-02 (WSP 70.7). In some examples, the vertical absorbency of the core material can vary from about 80 mm to about 150 mm, more particularly about 90 mm to about 130 mm, for example about 110 mm to about 125 mm or 120 mm. Vertical absorbency can be measured, for example, using a DIN 53106 test.

In some embodiments, the core material can be present as a substantially uniform sheet (as shown in FIG. 1) that covers a major portion of the backing layer 120. In other embodiments, the core material can be present in patches or strips disposed on the backing layer. For example, one or more patches or strips of core material can be disposed on the backing layer in different areas. If desired, the core material may also be configured as, or may include, one or more areas comprising a high friction surface material. Where a high friction surface material is present on the core material, it desirably does not interfere with the absorption of fluids by the core material.

In certain embodiments, the core material may comprise a desired pattern. For example, the pattern (if present) on the core material or core layers may vary depending on the desired aesthetic appearance and/or equipment used to produce the article. In some embodiments, one or more of a pinpoint pattern, square pattern, circular pattern, triangular pattern, rectangular pattern, hexagonal pattern or other geometric shapes may be present. In some instances as described herein, a pattern may be imparted to the core layer by using a roller comprising the particular pattern during manufacture of the article. In certain examples, the thickness of the overall article can vary from about 1 mm to about 2 mm, more particularly by about 1.2 mm to about 1.7 mm. In addition, the thickness of the article need not be uniform.

In other embodiments, additional materials can be present in the core layer, backing layer or both. For example, in certain embodiments one or more superabsorbent materials can be added, mixed with or otherwise disposed in the core material either prior to processing, during processing or after processing. Without wishing to be bound by any particular scientific theory, a superabsorber can act to absorb and/or retain large amounts of fluid. Illustrative superabsorbers are described, for example, in WO94/10596 and specific superabsorbers include, for example, acrylate composites, sodium polyacrylate, an acrylonitrile polymer, a polyacrylamide copolymer, an ethylene maleic anhydride copolymer, a cross-linked carboxymethylcellulose, a polyvinyl alcohol copolymer, a cross-linked polyethylene oxide, a starch grafted copolymer of polyacrylonitrile and other commonly available superabsorbent polymeric materials. In some embodiments, the process used to provide the core material may not include the use of any binders. In certain embodiments, the process used to provide the core material may be performed at room temperature and/or in ambient air. In certain examples, the core material can be produced as described in U.S. Pat. No. 6,675,702, the entire disclosure of which is incorporated herein by reference.

In one illustrative process for producing the article 100, a core material 110 can be produced by forming a web of material and coupling the formed web to a backing layer. In some embodiments, cellulose fiber comprising fluff pulp, e.g., dry wood pulp cardboards produced by means of a hammer mill, can be used to provide the core material. In one embodiment, a layer fibers, e.g., typically present in irregular or random orientations, can be conveyed to a first pair of calendar rollers on a strainer conveyer belt. One of the rollers can be heated or have a desired surface temperature, e.g., 200-220° C., while the other roller can remain unheated. If desired, the web can be moisturized, coated or sprayed with a desired substance prior to entering the gap between the two rollers. In some embodiments, the resultant moisture content of the material is about 5 to 10 percent by weight. During passage of the material between the rollers, the moisture content can be reduced from the use of the heated roller. After passage through the rollers, the cellulose fibers are compressed to provide a loose non-woven. The non-woven can be moisturized, coated or sprayed with a desired substance after exiting the rollers.

In certain embodiments, the loose non-woven can be provided to a pair of calendar rolls. The loose non-woven can be subjected to an array of point-shaped pressure zones, where the irregularly arranged fibers are pressed onto each other under high pressure, such that a close fusion of the fiber bodies occurs and a fiber web with a desired pattern can be provided that generally will not separate after the pressure is released. The particular pattern is not critical and illustrative patterns include pinpoint patterns, square patterns, circular patterns and other geometric patterns. The pressure used can result in “melting” of the fiber materials and provide close bonding. For example, through focused high pressure and crowding of the fibers, the loose cellulose or pulp fibers can be bonded together in free spaces resulting in an overall very strong fiber web. The calendar rolls can be operated at room temperature, e.g., between 18-25° C., but one or more of them may also be heated if desired. In some examples, the pressure provided by the calendar rolls can vary between 100 to 600 MPa, e.g., 500 MPa, but higher pressures can also be used. In certain examples, the resulting fiber web of material can comprise a basis weight, for example of about 50-1500 g/square meter. The fiber web exiting the calendars is significantly more tear resistant than the web entering the calendar rolls. In some examples, the resulting fiber web may then be provided to a drawing roller, and, if desired, wrapped onto a take-up roller with the use of a driver roller.

In certain examples, the loose material provided to the first set of rollers can be an inexpensive mass material that is available in large amounts. For example, fluff pulp with a whiteness of 85 to 89% can be used, which in turn means that a significant lignin and residue content is still present, which can assist in improving the bonding behavior. The fiber length of the material can vary and is desirably long enough such that they bridge the distance between the pressure zones. The additives described herein can be used, for example, in various amounts. In some instances, fluff pulp can be supplemented with superabsorbers with 0.5 to 70 percent in weight, preferably 5 to 30 percent in weight, and thereafter sent through the high-pressure calendar rolls. The superabsorbers generally have no bonding effect but become trapped in the non-woven once it is processed.

In certain embodiments, the core material can be produced without the use of any binding agents. By producing the core material without using any binding agents the recyclability and compostability of the product is improved. In addition, the production becomes less expensive and is simpler because stations for applying and curing are not required. In other embodiments, if desired, the core material can be produced using one or more binding agents or binding agents may be used to couple the core material to the backing layer and/or top layers as described herein.

In some embodiments, the finished core material may comprise numerous irregular cellulose fibers that are coupled by fusion in the pressure zones. The material itself can comprise a high tear strength and, in addition, a high absorption capacity, which is increased even further through the use of superabsorbers such that it can be used as packaging material, for hygiene articles other articles described herein.

In certain examples, the finished core material can be glued, welded, laminated or otherwise coupled to a backing layer, film or sheet. The backing layer may be pre-coated with an adhesive and coupled to the core material using one or more additional roller pairs to press the core material against the backing layer. Depending on the type of adhesive used, ultraviolet light, visible light, heat or other stimulus may be applied to the resulting composite to assist in curing of the adhesive.

In some embodiments, a material comprising a high coefficient of friction, e.g., 0.95 or greater, can then be laminated, coupled or otherwise joined to the backing layer to provide the resulting article with a coefficient of friction of 0.95 or greater as tested by ASTM D1894. In other embodiments, the backing layer can be coupled to the materials prior to coupling to the absorbent core material. In some instances, an entire sheet of material may be coupled to the backing layer either before or after coupling the backing layer to the absorbent core material.

In certain embodiments, the backing layer 120 can be a suitable material that can provide an article comprising the increased tear strength. While tear strength may be enhanced by the backing layer itself, it is desirable that the overall article provide the desired tear strength. For example, where a particular material is selected for use as a backing layer 120 in the article 100, it can be matched with a particular core layer 110 such that the overall article provides the desired tear strength. As one illustration, reinforcing materials can be added to the backing layer 120, the core layer 110 or both if the desired tear strength is not achieved or provided by each of the materials either alone or when combined. In some examples, the backing layer 120 can be produced using one or more thermosets. Where a thermoset is used, the thermoset may be present, for example, in a thin film such that the article 100 remains flexible and can be folded for packaging. In other embodiments, however, it may be desirable to provide or use a rigid backing layer 120 that remains substantially inflexible during use of the article. Illustrative thermoset materials include, but are not limited to, a polyester-fiberglass system, vulcanized rubber, a phenolic resin, a phenol-formaldehyde resin, a urea-formaldehyde foam, a melamine resin, a melamine-formaldehyde resin, an epoxy resin, a polyimide, a cyanate ester, a polycyanurate, a polyester thermoset such as, for example, an unsaturated polyester that can be cross-linked and other suitable thermoset materials and combinations of thermoset materials. If desired, the thermoset can be used with one or more cross-linking agents to facilitate setting of the material during processing. In other configurations, the thermoset can include one or more internal cross-linking sites which can be, under suitable conditions, cross-linked to other polymeric chains of the backing layer 120. In some embodiments, the backing layer 120 can comprise two or more different thermosets. In other embodiments, the backing layer 120 can comprise three, four, five, six, seven, eight or more different thermosets.

In other examples, the backing layer 120 can be produced using one or more thermoplastics or may include one or more thermoplastics. Thermoplastics provide desirable attributes including enhanced flexibility, desirable weight-to-strength ratios and easy processing. Illustrative thermoplastic materials include, but are not limited to, a polyethylene, a polypropylene, a polyester-polypropylene, an acrylonitrile butadiene styrene, a cellulose acetate, an ethylene-vinyl acetate, an ethylene vinyl alcohol, a fluoroplastic such as polytetrafluoroethylene, a polyoxymethylene, a polyacrylate, a polyacrylonitrile, a polyamide, a polyamide-imide, a polyetheretherketone, a polyaryletherketone, a polybutadiene, a polybutylene, a polycaprolactone, a polychlorotrifluoroethylene, a polyetherketoneketone, a polycarbonate, a polyhydroxyalkanoate, a polyketone, a polyester, a polyetherimide, a polysulfone, a polyimide, a polylactic acid, a polymethylpentene, a polyphenylene oxide, a polyphenylene sulfide, a polyphthalamide, a polystyrene, a polyurethane, a polyvinyl acetate, a polyvinyl chloride, a polyvinylidene chloride, a styrene-acrylonitrile, combinations thereof and other suitable thermoplastics. In some embodiments, the backing layer 120 can comprise two or more different thermoplastics. In other embodiments, the backing layer 120 can comprise three, four, five, six, seven, eight or more different thermoplastics. In certain embodiments, the backing layer 120 can comprise at least one thermoset and at least one thermoplastic material.

In certain embodiments, the backing layer can be, or can include, a non-woven material. For example, a non-woven material produced using a polyester and a polyethylene can be used as the backing layer 120. In other configurations, a non-woven material produced using a polyethylene and a polypropylene can be used as the backing layer 120. In additional configurations, a non-woven material produced using a polyester and a polypropylene can be used as the backing layer 120. In certain configurations, at least one of a polyethylene, a polypropylene, and a polyester is used in combination with another thermoplastic material to provide a backing layer 120 suitable for use in an article that provides the desired properties described herein, e.g., one that can be used with objects weighing 325 lbs or more without tearing.

In embodiments where a non-woven material is present as a backing layer 120, the non-woven material may be present as a substantially continuous sheet or, in some configurations, the non-woven material can be present in patches or otherwise separated by a different material. For example, it may be desirable to include the non-woven material in certain areas of the article 100 to increase the tear strength in those areas but use more flexible materials in other areas where increased strength is not needed or desired. In some examples, the non-woven material can be disposed on an additional backing layer or film in desired areas such that the composite backing layer can be used in the articles described herein.

In certain examples, the backing layer 120 may be present at a thickness from about 0.10 mm to about 1 mm, more particularly about 0.30 mm to about 0.80 mm. In some examples, the backing layer 120 may have a basis weight of about 30 grams/square meter to about 100 grams/square meter, e.g., 50 grams/square meter to about 80 grams/square meter. In some instances, where a non-woven material is present as backing layer 120, the basis weight of the non-woven material can be less than that of other commonly used materials while still providing the increased tear strength described herein. For example, where a non-woven material is present as a backing layer, the basis weight may be about 60 grams/square meter or less, whereas materials used in existing hygiene articles are typically present at a basis weight of about 70 grams/square meter or more.

In some embodiments, the backing layer 120 can be present as a continuous sheet comprising a substantially constant thickness throughout, whereas in other examples, the backing layer thickness may be variable to provide increased strength at certain areas, e.g., the edges, of the article 100. In some embodiments, the backing layer 120 may have decreased thickness in certain areas that couple to the high friction coefficient materials such that the overall thickness of the article is the same along the entire planar surface of the article. In examples where the article 100 is configured as one of many articles coupled to each other, the backing layer 120 can be perforated or include suitable features to permit rapid separation of the articles at a desired period.

In certain embodiments, the articles can be stacked onto each other or coupled to each other in a stack in a suitable manner. For example, a nurse or aid may stack a plurality of the articles onto each other and sequentially remove the top article as it becomes used by a patient or at periodic intervals. Where the articles are configured as a tuckable bed sheet, a plurality of bed sheets can be used one on top of the other and the top sheet can be removed as it becomes used. Where the articles are configured for use in animal cages, the articles can be stacked and coupled through the backing layer 120, e.g., through perforations in the backing layer 120, and the top article can be torn away after use to provide for a new article underneath. Other configurations of articles that can be stacked will be apparent to the person of ordinary skill in the art, given the benefit of this disclosure. The high friction surfaces of the backing layers of the articles described herein are particularly suited in settings where a human or other mammal may be stepping on, sliding on or moving over the article.

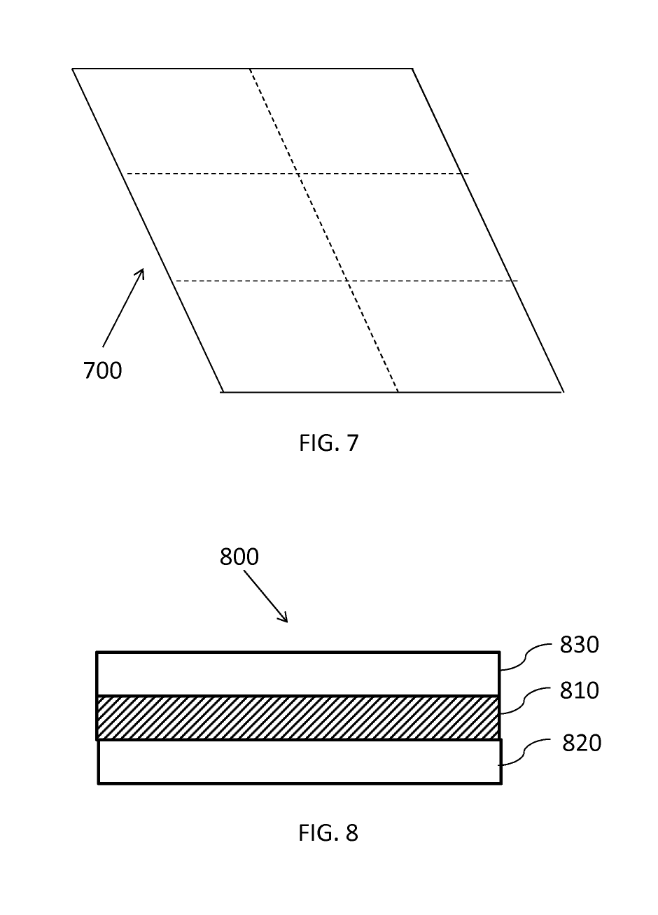

In certain examples, the backing layer 120 can be coupled to the core material 110 by pressing, rolling, using adhesives, using laser welding, melting or other processing steps as described herein. In some examples, a hot-melt adhesive can be placed between the backing layer 120 and the core material 110 and the resulting composite can be heated to couple the backing layer 120 to the core material 110. If desired, the composite can be passed through rollers, placed into a press or mold or otherwise a desired amount of pressure can be applied using suitable methods such as those described in U.S. Pat. No. 6,675,702, for example. Once the adhesive has cured, the composite sheet can be perforated in desired areas such that an article of a desired size may be produced by tearing or separation at the perforations. For example, in a typical operation a sheet of 2, 4, 6, 8 or more articles can be produced. Referring to FIG. 7, a plurality of articles, e.g., six, are shown as being present in a sheet 700. The articles can be separated along the perforation lines shows in FIG. 7. In some examples, the sheet 700 can be folded along the perforation lines to provide a smaller package for shipping purposes. If desired, the articles can be separated prior to packaging or may be separated by an end user prior to use. In some embodiments, the articles can be separated and packaged together in a desired amount, e.g., 5-10 articles. For example, a desired amount of articles can be wrapped together for purposes of sale or distribution.

In certain embodiments and referring to FIG. 8, a cross-section of another embodiment of an article is shown. The article 800 comprises a backsheet or backing layer 820, a core layer 810 and a top layer 830. As discussed herein, the backing layer 820 may comprise, or be configured as, a material that is effective to provide a coefficient of friction of greater than 0.95 as tested by ASTM D 1894. As also discussed in reference to FIG. 8, the term layer in reference to FIG. 8 is used herein for convenience purposes only and the various materials used in the article 800 can be configured as a film, sheet, patches or take other forms. In addition, while the layers are shown for illustration purposes in FIG. 8 as having the same thickness, it will be recognized by the person of ordinary skill in the art, given the benefit of this disclosure, that the layers 810, 820 and 830 typically will not have the same thickness. Illustrative thicknesses and dimensions for each layer are provided herein as representative sizes of the many different dimensions that can be present in the articles described herein. In addition, the term “top” is arbitrary and depends on a selected reference frame. Generally, a top layer refers to the layer closest to a patient or object during use of the article. In certain embodiments, the backing layer 820 can be selected from one or more polymeric materials as described herein, e.g., one or more non-woven materials. Similarly, the core layer 810 can comprise cellulose based materials and/or one or more superabsorbers as described herein.

In certain embodiments where a top layer is present, the weight of the top layer can vary from about 5 gsm to about 50 gsm, more particularly about 10 gsm to about 30 gsm, for example, about 10 gsm to about 25 gsm or about 15 gsm. In certain instances, the exact thickness of the top layer can also vary from about 0.10 mm to about 0.40 mm, for example, about 0.15 mm to about 0.30 mm or about 0.20 mm to about 0.25 mm. In some embodiments, the basis weight can be calculated by taking a known area of the pad or portion or component thereof, e.g., 0.5 square meters, and weighing it. The obtained mass can be divided by the area to obtain the weight in grams per square meter (gsm).

In certain examples, the tensile strength at break in the machine direction of the top layer can vary from about 10 N/5 cm to about 60 N/5 cm as tested using WSP 110.04(05) Option B Grip, Distance of 100 mm, more particularly about 25 N/5 cm to about 40 N/5 cm, for example about 30-40 N/5 cm. the tensile strength at break in the cross direction of the top layer can vary from about 5 N/5 cm to about 35 N/5 cm as tested using WSP 110.04(05) Option B Grip, Distance of 100 mm, more particularly about 10 N/5 cm to about 30 N/5 cm, for example about 15-20 N/5 cm.

In certain embodiments, the elongation at break in the machine direction of the top layer may be about 30% to about 100% as tested using WSP 110.04(05) Option B Grip, Distance of 100 mm, more particularly about 40% to about 80%, for example, about 50-60%.

In certain examples, the elongation at break in the cross direction of the top layer may be about 40% to about 110% as tested using WSP 110.04(05) Option B Grip, Distance of 100 mm, more particularly about 50% to about 80%, for example, about 60-70%.

In some embodiments, the liquid strikethrough of the top layer may be about 1 second to about 5 seconds as measured using WSP 70.3(05) test method, more particularly about 2 second to about 4 seconds, for example, about 2.5 seconds to about 3.5 seconds.

In other embodiments, the rewet of the top layer can vary from about 0.05 to about 1 grams as tested using WSP 80.10(05) test method, more particularly about 0.1 to about 0.25 grams, for example, about 0.15 grams or 0.20 grams or any value in between.

In certain embodiments, the top layer 830 may comprise a film, sheet or laminate of material that can be placed over the core layer 810 prior to pressurization of the core material or after pressurization of the core material. In some embodiments, the article 800 can be produced by disposing the top layer 830 over the core layer 810 and then embossing or providing a desired pattern in both the core layer 810 and the top layer 830. In some embodiments, the top layer 830 may only be present in selected areas such that the core layer 810 remains exposed in certain areas to ambient and can receive fluid.

In certain examples, the top layer 830 may comprise similar, or the same materials, as the backing layer 820, whereas in other examples, the top layer 830 may comprise different materials from the backing layer 820. In some examples, the top layer 830 may comprise one or more thermosets or thermoplastics as described herein. For example, where the top layer 830 comprises a thermoset, the thermoset may be, for example, a polyester-fiberglass system, vulcanized rubber, a phenolic resin, a phenol-formaldehyde resin, a urea-formaldehyde foam, a melamine resin, a melamine-formaldehyde resin, an epoxy resin, a polyimide, a cyanate ester, a polycyanurate, a polyester thermoset such as, for example, an unsaturated polyester that can be cross-linked and other suitable thermoset materials and combinations of thermoset materials. If desired, the thermoset can be used with one or more cross-linking agents to facilitate setting of the material during processing. In some examples, the top layer 830 can comprise two or more different thermosets. In other embodiments, the top layer 830 can comprise three, four, five, six, seven, eight or more different thermosets. Where the top layer comprises a thermoplastic, the thermoplastic may be, for example, a polyethylene, a polypropylene, a polyester-polypropylene, an acrylonitrile butadiene styrene, a cellulose acetate, an ethylene-vinyl acetate, an ethylene vinyl alcohol, a fluoroplastic such as polytetrafluoroethylene, a polyoxymethylene, a polyacrylate, a polyacrylonitrile, a polyamide, a polyamide-imide, a polyetheretherketone, a polyaryletherketone, a polybutadiene, a polybutylene, a polycaprolactone, a polychlorotrifluoroethylene, a polyetherketoneketone, a polycarbonate, a polyhydroxyalkanoate, a polyketone, a polyester, a polyetherimide, a polysulfone, a polyimide, a polylactic acid, a polymethylpentene, a polyphenylene oxide, a polyphenylene sulfide, a polyphthalamide, a polystyrene, a polyurethane, a polyvinyl acetate, a polyvinyl chloride, a polyvinylidene chloride, a styrene-acrylonitrile, combinations thereof and other suitable thermoplastics. In some embodiments, the top layer 830 can comprise two or more different thermoplastics. In other embodiments, the top layer 830 can comprise three, four, five, six, seven, eight or more different thermoplastics. In certain embodiments, the top layer 830 can comprise at least one thermoset material and at least one thermoplastic material.

In certain embodiments, the top layer 830 can be produced using a polyester and a polyethylene. In other configurations, the top layer 830 can be produced using a polyethylene and a polypropylene. In additional configurations, a polyester and a polypropylene can be used as the top layer 830. In certain configurations, at least one of a polyethylene, a polypropylene, and a polyester is used in combination with another thermoplastic material to provide a top layer 830 suitable for use in an article that provides the desired properties described herein.

In certain embodiments, by selecting and combining a backing layer, core material and a top layer each comprising suitable materials, the articles described herein can provide the desired coefficient of friction while still functioning as an absorbent article. In particular, any one or more materials by themselves may not necessarily provide the desired functionality, but when the backing layer and core material layer (and optionally the top layer) and coupled to each other, the resulting article can provide the desired physical properties.

In certain examples, the top layer 830 can include reinforcing materials, pigments, colorants, or other additives to provide desired physical properties or aesthetic features. For example, the top layer can include fibers, whiskers, powders, reinforcing materials, colorants, markings, lettering or other suitable features. In some embodiments, the top layer may comprise a basis weight of about 5 grams/square meter to about 30 grams/square meter, e.g., about 10 grams/square meter to about 22 grams/square meter. Where a top layer 830 is present, it may be present as strips, patches or as a continuous sheet disposed on the core material layer. In addition, in some examples different areas of the article may include top layers comprising different materials. For example, it may be desirable to include a top layer with a higher tear strength near the edges of the article and a top layer with a lower tear strength in central portions of the article. In other configurations, the thickness of the top layer may vary at different portions of the article.

In certain embodiments, one or more strips of material may be added to cover portions of the article where the top layer is absent. For example, it may be desirable to leave a portion of the core exposed to the surface, e.g., not to include a top layer on certain areas, and such exposed areas can be covered with another material if desired.

In certain instances, a cellulose based material such as tissue, pulp or the like can be placed over the exposed areas. In some embodiments, two or more different materials can be added to the exposed areas. The material can be added in the form of a sheet, strips, segments or the like. In certain embodiments, the basis weight of the material which is added can vary from about 7 lbs/3000 ft² to about 15 lb/3000 ft², more particularly about 9 lbs/3000 ft² to about 13 lb/3000 ft², for example about 10.5 to about 11.5 15 lb/3000 ft². In other examples, the tensile strength of the added material (when dry) in the machine direction can vary from about 500 grams/inch to about 750 grams/inch, for example about 500 grams/inch to about 650 grams/inch or about 550 grams/inch or about 525 grams/inch to about 625 grams/inch. In some examples, the tensile strength of the added material (when wet) in the machine direction can vary from about 75 grams/inch to about 150 grams/inch, for example about 85 grams/inch to about 125 grams/inch or about 90 grams/inch or about 115 grams/inch to about 625 grams/inch. In other embodiments, the stretch at break in the machine direction of the added material can vary from about 20% to about 40%, more particularly about 25% to about 35%, for example about 30%. In additional embodiments, the porosity of the added material can vary from about 175 to about 275 ft³/min-ft², more particularly about 190 to about 250 ft³/min-ft², for example about 200 to about 250 ft³/min-ft². The brightness of the added material can vary and illustrative values include, but are not limited to, 70-100 GE brightness units, more particularly about 80-95 GE brightness units, for example, about 80-90 GE brightness units. The moisture content of the added material may be from about 2% to about 10%, more particularly from about 3% to about 9%, for example, about 4-6%.

In certain embodiments where a top layer is present or where material is added to the article at areas of the core that are exposed, an adhesive can be used to adhere the top layer or material to the core. In some embodiments, the adhesive selected for use desirably does not interfere with the physical properties of the core material. In some embodiments, a thermoplastic adhesive can be used to couple the top layer or added material to the core layer. In other embodiments, a thermoset adhesive can be used to couple to the top layer or added material to the core layer. In additional embodiments, a mixture of a thermoplastic/thermoset adhesive can be used to couple the top layer or added material to the core layer. In some examples, the adhesive may be a polyolefin adhesive. The physical properties of the adhesive can vary and desirably the adhesive provides an effective adhesive strength to retain the top layer and/or added material to the core layer. In some embodiments, the adhesive may have a density close to that of water, e.g., about 1 g/cm³, whereas in other embodiments, the adhesive may have a density that ranges from about 0.8 g/cm³ to about 1.25 g/cm³.

In certain embodiments, the overall thickness of the articles can vary depending on the intended use of the article. In some embodiments, the articles may be designed to be thin, e.g., 2 mm or less, whereas in other examples it may be desirable to increase the overall thickness of the article to provide for increased absorption, for example. In some embodiments, the thickness of the article can vary from about 1 mm to about 20 mm, more particularly, from about 1.5 mm to about 10 mm, for example, about 1.5 mm to about 9 mm, about 1.75 mm to about 5 mm or any value within these illustrative ranges.

In certain embodiments, the overall dimensions, geometry and shape of the article can vary. In some embodiments, the article can take the form of a pad with a width of about 15 inches to about 45 inches, more particularly a width of about 20 inches to about 40 inches, e.g., about 30-35 inches. In certain configurations, the pad may be about 20 inches to about 90 inches long, more particularly about 25 inches to about 80 inches long, for example about 30-40 inches or about 40-65 inches. In certain examples, the length of the core layer in the pad can be less than the overall length of the pad. For example, the core layer can be about 25 inches to about 60 inches, more particularly about 30 inches to about 50 inches, for example about 30 inches to about 45 inches.

In certain embodiments, the articles described herein can include one or more antimicrobial materials coated, sprayed or otherwise disposed on them. In some examples, the antimicrobial material may be present on the top layer (if present), whereas in other examples, the antimicrobial material may be present on the core layer or other layers if present. If desired, the backing layer may also include an antimicrobial material. The antimicrobial material may be added to the various materials of the article prior to processing, during processing or after processing.

In certain embodiments, the antimicrobial material may be an antibacterial material, an antiviral material, an antifungal material, an antiparasitic material or combinations thereof. Illustrative antibacterial materials include, but are not limited to, those that target one of more of the bacterial cell wall, the bacterial cell membrane, or bacterial protein synthesis, a bacterial enzyme inhibitor, a sulfonamide, a trimethoprim-sulfamethoxazole, a quinoline, a penicillin, a cephalosporin, a Beta-lactam, a Beta-lactamase inhibitor, an aminoglycoside, streptomycin, gentamicin, tobramycine, amikacin, netilmicin, kanamycin, neomycin, a tetracycline, chloramphenicaol, a macrolide, erythryomycin, vancomycin, clindamycin, spectinomycin, polymyxin B, colistin, teicoplanin, bacitracin, a glycylcycline, isoniazid, rifampin, triclosan, ethambutol, pyrazinamide, ethionamide, aminosalicyclic acid, clofamizine, a quinolone, a sulfone and other antibiotics commonly administered topically or systemically to humans or other mammals to treat a bacterial disease or infection.

Illustrative antiviral materials include, but are not limited to, those that interfere with viral development or stabilization, e.g., those that disrupt viral nucleic acid or viral protein coats and those commonly used to treat both viral and retroviral diseases in humans. Additional illustrative antiviral materials include, but are not limited to, a uridine based drug, acyclovir, valacyclovir, famciclovir, penciclovir, foscarnet, ganciclovir, idoxuridine, sorivurdine, trifluridine, vidarabine, zidovudine, didanosine, stavudine, zalcitabine, amantadine, rimantadine, an interferon, a polyclonal antibody, a monoclonal antibody, lamivudine, ribavirin, a protease inhibitor, an acyclic nucleoside phosphonate and other commonly used viral and retroviral drugs administered topically or systemically to humans or other mammals to treat viral diseases or infections.

Illustrative antifungal materials include, but are not limited to, a polyene antifungal, an imidazole, a triazole, a thiazole, an allylamine, an echinocandin, an amphotericin, flucytosine, ketoconazole, micanozole, itraconazole, fluconazole, griseofulvin, terbinafine, nystatin, Lamisil or other antifungals commonly applied topically or taken systemically by humans or other mammals to treat fungal diseases or infections.

Illustrative antiparasitic materials include, but are not limited to, a drug used to treat a protozoan infection, a drug used to treat a metazoan infection, a schizontocide, a gametocytocide, a sprontocide, a quinone, a haloquinone, a chloroquinone, a quinoline, a haloguanide, a chloroguanide, a diaminopyrimidine, a halofantrine, a mefloquine, a primaquine, quinine, atovaquone, diloxanide furoate, eflornithine, melarsoprol, emetine, dehydroemitine, metronidazole, nifurtimox, pentamidine, quinacrine, sodium stibogluconate, suramin, paramoycin, a benzimadozole, a diethylcarbamazine, a permectin, metrifonate, niclosamide, oxamniquine, piperazine, praziquantel, pyrantel pamoate, a sulfonamide, a sulfone, a tetracycline and other drugs commonly administered topically or systemically to humans or other mammals to treat parasitic and helminthic infections.

In certain examples where an antimicrobial material is present, a removable liner or sheet may be present on the top of the article to prevent exposure of the antimicrobial to the environment prior to use. The liner may comprise paper, plastics or other materials that are generally lightweight, can be sterilized and/or are easily removable from the article prior to use. In some configurations, the liner desirably does not remove substantial amounts of the antimicrobial material from the article when the liner is removed. If desired, the antimicrobial may be heat, light, pressure or moisture activated such that it is generally non-active until a suitable perturbation is applied. In some examples, the antimicrobial material can be added to the article using many different methods. For example, it can be sprayed on, coated on, brushed on, rolled on, impregnated in, present in encapsulated form such that pressure by a patient or object bursts the capsules and exposes the antimicrobial, or may be added in other form and using other methods.

In certain embodiments, the antimicrobial material can be present in an effective amount such that some percentage of microbial growth or reproduction is inhibited. In some examples, the antimicrobial may be present in an amount effective to reduce microbe levels by 10%, 20%, 30%, 40%, 50%, 60%, 70%, 80%, 90% or more when compared to a similar article that lacks the antimicrobial.

In some examples, the article can include an insect repellent or an animal repellent to deter insects or animals from being on or near the article. For example, N,N-Diethyl-meta-toluamide (DEET), picaridin, citronella oil, coal tar extracts or other materials may be disposed on the article or impregnated in the article to deter insects and/or animals from approaching or remaining near the article. If desired, an insecticide, fungicide, herbicide, etc. may also be present on the article to kill unwanted organisms that may come in contact with the article.

In certain embodiments, the articles described herein can be sterilized prior to, during or after packaging. Many different sterilization methods can be used and desirably, non-moisture based sterilization methods are used so that the core material is not unnecessarily exposed to moisture prior to use. Illustrative types of sterilization methods include, but are not limited to, gamma radiation, electron beam radiation, X-ray radiation, ultraviolet radiation, ozonation, ethylene oxide gas exposure and other suitable non-water based sterilization methods. In some examples, the articles may be packaged in plastic or paper bags or receptacles, sealed from the ambient and then sterilized using one or more suitable methods and materials. In other examples, sterilization may take place prior to packaging or immediately prior to use of the article.

In certain examples, the articles described herein can include cosmetic agents. For example, the core material, top layer (when present) or both can include one or more cosmetic agents designed to provide a desired effect. Illustrative cosmetic agents include, but are not limited to, a moisturizer, a perfume, a sunblock, an exfoliant, a lotion, a powder, a polish, a sanitizer, a salt, a butter, a skin lightener, an anti-acne agent, an anti-wrinkle agent, a tanning agent, an oil, or other suitable cosmetic agents commonly applied to the skin of humans. In some examples, the article can be used with a cosmetic agent once the cosmetic agent has been applied to the skin or other desired area. For example, the cosmetic agent can be applied to the skin and then an article can be applied over the same area of the skin. In other examples, the article can be first applied to the skin to remove any unwanted moisture and then a cosmetic agent may be applied optionally followed by application of another article. For example, it may be desirable to apply the article to burn patients to remove residual moisture from the areas prior to application of desired cosmetic or therapeutic agents.

It will be recognized by the person of ordinary skill in the art, given the benefit of this disclosure, that the articles described herein can be configured or sized and arranged in many different forms, shapes and arrangements. In one embodiment, the articles described herein can be sized and arranged for use as a table pad or a gurney pad. For example, the dimensions of the article may be similar to, or the same as, the upper surface of a table or gurney so that the article covers substantially the top surface of the table or gurney. The article can be placed on the table or gurney and generally held in place through the high friction surface of the article.

In certain embodiments, the articles described herein can be sized and arranged to be placed in the seat of a wheelchair. In use, the article is typically inserted into the seat of the wheelchair prior to placement of the subject in the wheelchair. The increased friction of the article can act to retain the article in place as the subject is placed into the wheel chairs.

In certain examples, the articles described herein can be used as liners for radiological instruments. For example, the non-magnetic nature of certain embodiments of the articles described herein permits use with magnetic resonance (MR) imaging devices without the risk of interfering with the MR scans. Similarly, the articles described herein can be configured as radio-transparent articles such that X-rays may pass through the articles without substantial interference from the articles. By using the articles herein with radiological instruments, the possibility of fluid contamination of the instrument is reduced without substantial interference by the article. In addition, the articles permit usage with large patients, e.g., those weighing 325 lbs or more, during radiological tests on the large patients.

In certain embodiments, the articles used herein can be configured as a sanitary pad. For example, the article can be configured to receive menses, post-partum fluids, post-hysterectomy fluids, fluids excreted following bladder repair or vaginal repair procedures or other fluids commonly excreted by females. The increased friction permits use of the articles without the need to use an adhesive. For example, female sanitary pads can be held in place in underwear without the need to include an adhesive on one or more surfaces.

In some embodiments, the articles described herein can be configured as a nursing pad. In some configurations, the nursing pad can be designed to be placed into a nursing bra or other bra to absorb breast milk that may leak or be excreted from the breast. The high friction surface of the nursing pad can permit the pad to be held in place without the use of any fastener or releasable adhesive on one or both sides to assist in retention of the nursing pad in a desired spot in the bra. In other configurations, the articles described herein permits construction of an entire bra comprising areas of the superabsorbent material. For example, a disposable bra that can be used in a hospital setting for mothers can be constructed with areas of the bra near or adjacent to the nipple area comprising the superabsorbent core material for absorption of breast milk. The bra can be designed for wearing for a desired period, e.g., 1-3 days, and then may be disposed of and replaced with a new bra.

In other embodiments, the articles described herein can be configured as a surgical pad effective to absorb fluids such as blood during a surgical procedure. For example, the article can be used to blot fluids during surgery to increase the visual access by a surgeon to desired areas. If desired, the article can be used as a surgical wrap post-surgery to reduce the amount of moisture present at an incision site and reduce the overall risk of infection. In some embodiments, the wrap may comprise a self-sticking backing layer such that as the article is wrapped around a patient it sticks to itself and is retained without the use of clips or tape. If desired, however, clips, tape or other means can be used to hold the wrap in place.

In certain examples, the articles described herein can be configured as a diaper, e.g., a baby diaper or an adult diaper. In some embodiments, the core material of the diaper may only be present in areas likely to be exposed to liquid such as urine, whereas in other configurations, the core material can be present throughout the diaper. In some instances the diaper shell may include insertable areas where the article can be placed to absorb fluids. Such areas are typically at areas of the diaper near the front, back and the portion running between the legs, though insertable areas can be positioned in other areas as well.

In certain embodiments, the articles described herein can be used in or configured as disposable underwear. For example, pregnant women typically are provided disposable underwear during labor. The articles described herein can be used in a similar manner and may include perforations at the sides to permit tearing or removal when desired. The underwear may be configured in various sizes to accommodate different dimensions, weights and/or body types.

In certain embodiments, the articles described herein can be configured as a tuckable sheet, e.g., one sized and arranged for use in a standard hospital bed. For example, the sheet can be sized to fit onto a standard hospital bed of about 36 inches wide by 80 inches long and include tuckable portions, e.g., elastomeric or flexible portions, at each corner to facilitate retention of the sheet on the hospital bed. In certain embodiments, the tuckable sheet comprises a backing layer and a core material disposed on the backing layer, in which the tuckable sheet is configured with a surface comprising a coefficient of friction of about 0.95 or more as tested by AST?M D 1894. The high friction surfaces of the tuckable sheet can hold the sheet in place as a patient is moved into or out of the bed. In some embodiments, the tuckable sheet can include a core material throughout the surface positioned on the hospital bed, whereas in other configurations, the core material can be present in select areas of the tuckable sheet. For example, it may be desirable to only include the core material in the middle portion of the tuckable sheet where a patient's mid-body and upper legs would rest. In some configurations, the tuckable sheet may comprise replaceable sections of core material such that as the core material is exposed to fluids, the used core material may be replaced with a fresh section without having to replace the entire tuckable sheet. In other embodiments, the tuckable sheet can be stacked or used with additional tuckable sheets so that after use the top sheet can be removed exposing a new sheet.

In certain examples, the articles described herein can be configured as a wearable garment such as, for example, a shirt or other clothing. In some embodiments, the article may be configured as a disposable base layer effective to absorb sweat from a subject. For example, athletes, hikers or other active individuals can use the garment as a layer placed directly in contact with the skin. The garment can remove moisture and sweat from the skin to assist in keeping the subject feeling warm and dry.

In some examples, the wearable garment can be configured as a hospital gown. For example, the lower front portion, back portion or both of the hospital gown can comprise the superabsorbent material to absorb urine, sweat or other body fluids of the patient. If desired, the gown may include side flaps or snaps to facilitate easy removal of the gown from the patient. The gown may be made entirely from the article or may only include areas comprising the article. In other configurations, a conventional hospital gown comprising a pocket or flap configured to receive one or more of the article described herein can be used. For example, an article can be inserted into the pocket, and once used, can be removed from the pocket and replaced with a new article. Additional configurations of using the articles described herein as or with hospital gowns will be readily selected by the person of ordinary skill in the art, given the benefit of this disclosure.

In certain embodiments, the articles described herein can be configured as an insert for a hat or helmet to absorb sweat or other bodily fluids. For example, the articles can be inserted into athletic helmets, construction helmets, military helmets or other helmets commonly worn to provide for absorption of sweat from the wearer's head. In some examples, the insert may take the form of a liner that cover substantially all the inner surfaces of the helmet, whereas in other examples, the insert may take the form of small patches that can be positioned at desired areas, e.g., the forehead, temples or both. The high friction surface of the article can assist in retaining the headwear on the head.

In some embodiments, the wearable garment may take the form of an insole or shoe insert. The high friction surfaces of the articles permit their use as insoles without sliding of the insole in the shoe. If desired, one or more similar high friction surfaces may be present that can contact the foot or a sock to prevent movement of the foot within the shoe. If desired, the insoles or inserts can be designed as single use disposable items to absorb foot moisture. For example, the insole can be used for a single day and then replaced with a new insole the next day to provide a fresh surface to absorb foot moisture.

In certain examples, the articles described herein can be used to absorb fluids from surfaces to prevent growth of mold, bacteria or other organisms of the surfaces. For example, the articles can be placed in food packaging to absorb water or moisture within the package to prevent the food from being exposed to the water. In some examples, the articles may be placed in shipping crates, plastic bags or other forms of packaging commonly used to ship and/or sell food products such as produce, meats, cheeses or other foods. The high friction surfaces of the articles described herein permit use of the articles to absorb moisture while reducing movement of the articles as the packages are moved.

In certain embodiments, the articles described herein can be used to reduce and/or alleviate pressure sores. In some examples, the core material can relieve pressure points and can act to absorb fluids from areas having pressure sores to reduce the likelihood of infection at the pressure sore sites. In embodiments where the articles comprise an antimicrobial material, the antimicrobial material may also assist in reducing or alleviating pressure sores by slowing or preventing growth of microorganisms at the pressure sore sites.

In certain examples, the articles described herein can be used in agricultural settings. For example, the article can be used as liners in animal stalls or cages. The increased tear resistance of certain embodiments of the articles disclosed herein lowers the likelihood that the article will be torn from chewing by the animals or from being stepped on. In addition, the high friction surfaces permits their use in horse stalls or other animals stalls or cages without worrying about the animals slipping from movement of the articles underneath them. In some examples, the article can be sized and arranged such that it fits the floor of the cage or stall. In other agricultural uses, the articles can be used in meat processing facilities to absorb blood, dry meat or during transport of meat from one processing station or site to another.

In some examples, the articles described herein can be used in an industrial setting to absorb excess moisture or for other uses. In some embodiments, the article may be saturated with water and then used as a covering to slow diffusion of water from a substrate, e.g., concrete, brick or other building materials. By including a high friction surface, the article can be placed and generally is held in place and resistant to movement from wind or other forces. In other embodiments, the article may be placed on the substrate to increase the rate of diffusion or lower the drying time of the substrate. In additional embodiments, the article may be used to wrap building materials to reduce the likelihood of the building materials absorbing water and possibly becoming moldy prior to use. Other industrial uses of the articles described herein will be recognized by the person of ordinary skill in the art, given the benefit of this disclosure.

In some embodiments, the articles described herein can be used as liners for shelves in pantries, refrigerators or other areas where it may be desirable to remove moisture. For example, sections of the article may be cut or the article may be sized and arranged to be inserted into crisper drawers or placed on refrigerators shelves to absorb excess moisture from food products in the refrigerator. In some examples, the article can be used with an insert designed to retain the article in place or the shelf or drawer may include fitting, tabs or holder to hold the article in place during use.

In certain embodiments, the components of the articles described herein can be provided in kit form. For example, a backing layer, core material and optionally a top layer can be separately provided and used to provide an article. An end user can assemble the core material to the backing layer using a suitable adhesive, a mold or press or other devices designed to assist in coupling of the backing layer to the core material. In some examples, the kit can include instructions for coupling the backing layer to the core material such that a user may assemble the article on-site. In some configurations, the kit may also include a roller or rollers, a press, mold or other devices designed to apply pressure to the core material and/or backing layer. In certain instances, the kit can include an adhesive which may be used to couple the backing layer to the core material. In other configurations, the kit may include a hot air gun which can be used to melt the adhesive and couple the core material to the backing layer. Additional components may also be included in the kit to provide an article suitable for an intended use.

In certain examples, a method of facilitating production of an article comprising providing a backing layer effective to provide a coefficient of friction that is greater than 0.95 as tested by ASTM D 1894, and providing instructions for using the backing layer with a core material to provide an article is provided. In some embodiments, the method comprises providing the core material. In certain embodiments, the method comprises providing a top layer material configured to couple to the core material. In certain examples, the method comprises providing instructions for forming a pattern in the article. In other examples, the method comprises providing an antimicrobial material for applying to the article. In certain embodiments, the method comprises configuring the article to comprise an effective tear strength to be removed from under a patient weighing about 325 lbs or more without substantial tearing of the article. In other instances, the method comprises providing a plurality of different core materials. In some instances, the method comprises providing a plurality of different backing layers each effective to provide the coefficient of friction that is greater than 0.95 as tested by ASTM D 1894. In certain examples, the provided backing layer comprises a strip of material that provides the coefficient of friction that is greater than 0.95 as tested by ASTM D 1894. In some embodiments, the provided backing layer comprises a unitary sheet that provides the coefficient of friction that is greater than 0.95 as tested by ASTM D 1894.

When introducing elements of the aspects, embodiments and examples disclosed herein, the articles “a,” “an,” “the” and “said” are intended to mean that there are one or more of the elements. The terms “comprising,” “including” and “having” are intended to be open-ended and mean that there may be additional elements other than the listed elements. It will be recognized by the person of ordinary skill in the art, given the benefit of this disclosure, that various components of the examples can be interchanged or substituted with various components in other examples.

Although certain aspects, examples and embodiments have been described above, it will be recognized by the person of ordinary skill in the art, given the benefit of this disclosure, that additions, substitutions, modifications, and alterations of the disclosed illustrative aspects, examples and embodiments are possible.

1. An article comprising an absorbent core material disposed on a backing layer, in which the backing layer comprises at least one area comprising a coefficient of friction that is greater than 0.95 as tested by ASTM D 1894 dated 2008.

2. The article of claim 1, in which the article comprises an effective tear strength to be removed from under an object, weighing about 325 lbs or more, on the article without substantial tearing of the article.

3. The article of claim 2, in which the backing layer comprises a non-woven material effective to provide the effective tear strength to the article.

4. The article of claim 1, in which the absorbent core material comprises at least one superabsorber.

5. The article of claim 3, further comprising a top layer disposed on the absorbent core material.

6. The article of claim 3, in which the top layer comprises a polypropylene material.

7. The article of claim 5, in which at least one section of the absorbent core material is exposed to the environment.

8. The article of claim 5, further comprising a tissue paper layer covering adjacent sections of the absorbent core material.

9. The article of claim 1, in which the backing layer comprises two substantially parallel strips to provide the coefficient of friction that is greater than 0.95 as tested by ASTM D 1894.

10. The article of claim 1, in which the entire backing layer comprises a unitary sheet effective to provide the coefficient of friction that is greater than 0.95 as tested by ASTM D 1894.

11. An article comprising an absorbent core material disposed on a backing layer, in which the article is sized and arranged to be placed under a human to absorb bodily fluids from the human and in which the article comprises at least one material disposed on the backing layer that is effective to provide a coefficient of friction that is greater than 0.95 as tested by ASTM D 1894 dated 2008.

12. The article of claim 11, in which the at least one material disposed on the backing layer is configured as a plurality of strips.

13. The article of claim 11, in which the at least one material disposed on the backing layer is configured as a sheet.

14. The article of claim 11, in which the absorbent core material comprises at least one superabsorber.

15. The article of claim 14, further comprising a top layer disposed on the absorbent core material.

16. The article of claim 14, in which the top layer comprises a polypropylene material.

17. The article of claim 16, in which at least one section of the absorbent core material is exposed to the environment.

18. The article of claim 16, further comprising a tissue paper layer covering adjacent sections of the absorbent core material.

19. The article of claim 11, in which the at least one material disposed on the backing layer comprises a plurality of offset strips.

20. The article of claim 11, in which the at least one material disposed on the backing layer comprises at least two areas with different coefficients of frictions greater than 0.95 as tested by ASTM D 1894.

21-120. (canceled)
